(19) United States
(12) Patent Application Publication
(10) Pub. No.: US 2026/0137385 A1
(43) Pub. Date: May 21, 2026

(54) SUTURE PASSER AND METHOD OF TISSUE REPAIR

(71) Applicant: Arthrex, Inc., Naples, FL (US)

(72) Inventor: Jason A. VALENTIN, Fort Myers, FL (US)

(21) Appl. No.: 19/397,392

(22) Filed: Nov. 21, 2025

Related U.S. Application Data
(60) Provisional application No. 63/723,519, filed on Nov. 21, 2024.

(51) Int. Cl. A61B 17/04; A61B 17/06

(52) U.S. Cl. CPC A61B 17/0491; A61B 17/0469; A61B 17/06066; A61B 17/06166

(57) ABSTRACT

A suturing apparatus may include a shaft member having a lumen, and a first jaw member and a second jaw member that move between an open configuration and a closed configuration. A distal portion of the first jaw member may be pivotably connected to a distal portion of the second jaw member, and the distal portion of the second jaw member may have a distal opening. The suturing apparatus may further include a needle translates through the lumen of the shaft member and carries a suture, and a retention member in the distal opening of the second jaw member to remove the suture from the needle. The suture may be wrapped around the distal portion of the second jaw member. The suturing apparatus may be included in a suturing system having a suture. The suturing apparatus may be used in a method of repairing tissue.

### CROSS REFERENCE TO RELATED APPLICATIONS

This application claims the benefit of U.S. Provisional Patent App. No. 63/723,519, filed Nov. 21, 2024, the disclosure of which is hereby incorporated by reference herein.

### TECHNICAL FIELD

The disclosure relates to the field of surgery and, more specifically, to suturing instruments and methods of passing suture.

### BACKGROUND

When a tendon becomes damaged, physicians often perform procedures including a tenotomy or a tenodesis to repair an attachment of the tendon to a bone tissue. The procedures often involve suturing the tendon and anchoring the suture to the bone tissue.

### SUMMARY

Current techniques of securing a tendon to bone tissue can involve complex suturing that can be difficult to perform in the minimal intracorporeal space. The limited space often necessitates performing the suturing outside of the body and/or introducing additional ports for improved access. The present invention provides an improved, simplified technique that facilitates the securing of the tendon through a tenotomy or a tenodesis, among other surgical procedures.

Thus, a first aspect of the present disclosure is directed to a suturing apparatus comprising: a shaft member having a lumen; a first jaw member and a second jaw member configured to move between an open configuration and a closed configuration, wherein a distal portion of the first jaw member is pivotably connected to a distal portion of the second jaw member, and the distal portion of the second jaw member has a distal opening; a needle configured to translate through the lumen of the shaft member and configured to carry a suture; and a retention member in the distal opening of the second jaw member, wherein the retention member is configured to engage the suture and remove the suture from the needle.

The suturing apparatus of the first aspect may have one or more of the following features. The suturing apparatus may have a handle assembly; and a cleat on a first side of the shaft member and/or the handle assembly, wherein the suture is configured to be wrapped around the distal portion of the second jaw member under tension and engage the cleat to maintain the tension. The suturing apparatus may have a second cleat on a second side of the shaft member and/or the handle assembly opposite of the first side, wherein the second cleat is configured to engage the suture. The suturing apparatus may have a spring configured to bias the retention member in a closed configuration. The spring may be a separate component from the retention member, and the spring may be a helical spring or a leaf spring. The suture may be configured to be wrapped around the distal portion of the second jaw member on a lateral side of the retention member. The distal portion of the second jaw member may have at least one distal channel on the lateral side of the retention member configured to receive the suture. A proximal portion of the first jaw member may have a lateral channel configured to receive the suture, and a longitudinal channel configured to receive the needle. The second jaw member may have a proximal portion having a seat configured to receive the suture. The needle may have a groove between a first prong and a second prong, and the groove may be configured to receive the suture. A suturing system may include the suture apparatus; and the suture having a loop and a tail, wherein the loop of the suture may be engaged by a proximal portion of the first jaw member, the suture may be wrapped around the distal portion of the second jaw member, and the needle may engage a length of the tail and feed the length of the tail through the loop of the suture.

A second aspect of the present disclosure is directed to a suturing apparatus comprising: a shaft member having a lumen; a first jaw member and a second jaw member configured to pivot between an open configuration and a closed configuration, wherein a distal portion of the second jaw member has a distal opening; a needle configured to translate through the lumen of the shaft member and configured to carry a suture; and a retention member in the distal opening of the second jaw member, wherein the retention member is configured to engage the suture and remove the suture from the needle, wherein a distal surface of the first jaw member has at least one distal channel configured to receive a length of the suture.

The suturing apparatus of the second aspect may have one or more of the following features. The first jaw member may be configured to pivot in a lateral plane relative to the second jaw member, and the at least one distal channel extends along the lateral plane. The at least one distal channel may include a first distal channel on a first lateral side of the distal opening and a second distal channel on a second lateral side of the distal opening. A distal portion of the first jaw member may be pivotably connected to a distal portion of the second jaw member. A proximal portion of the first jaw member may have a lateral channel configured to receive the suture, and a longitudinal channel configured to receive the needle. The second jaw member may have a proximal portion having a seat configured to receive the suture. The retention member may be configured to pivot relative to the first jaw member. The suturing apparatus may have a spring configured to bias the retention member in a closed configuration. A suturing system may include the suturing apparatus; and the suture having a loop and a tail, wherein the loop of the suture may be engaged by a proximal portion of the first jaw member, the suture may be wrapped around a distal portion of the second jaw member, and the needle may engage a length of the tail and feed the length of the tail through the loop of the suture.

A third aspect of the present disclosure is directed to a suturing system comprising: a suture; a suturing apparatus comprising: a shaft member having a lumen; a first jaw member and a second jaw member configured to pivot between an open configuration and a closed configuration, wherein a distal portion of the first jaw member is connected to a distal portion of the second jaw member; and a needle configured to translate through the lumen of the shaft member and configured to carry the suture, wherein the suture is engaged by the first jaw member, the suture is wrapped around the distal portion of the second jaw member.

The suturing apparatus of the third aspect may have one or more of the following features. The suture may have a loop and a tail, wherein the loop of the suture may be engaged by a proximal portion of the first jaw member, and the needle may engage a length of the tail and feed the length of the tail through the loop.

A fourth aspect of the present disclosure is directed to a suturing system comprising: a suture having a loop and a tail; a suturing apparatus comprising: a shaft member having a lumen; a needle configured to translate through the lumen of the shaft member; a first jaw member and a second jaw member configured to move between an open configuration and a closed configuration, wherein a distal portion of the first jaw member is pivotably connected to a distal portion of the second jaw member, and the distal portion of the second jaw member has a distal opening; and a retention member in the distal opening of the second jaw member, wherein the retention member is configured to engage the suture and remove the suture from the needle, wherein the loop of the suture is engaged by a proximal portion of the first jaw member, and a length of the tail is positioned at an opening of the lumen of the shaft member, wherein the first jaw member, when in the closed configuration, is configured to align an opening of the loop with the opening of the lumen, the needle is configured to engage the length of the tail and feed the length of the tail through the loop, and the retention member is configured to secure the length of the tail.

A fifth aspect of the present disclosure is directed to a method comprising: receiving a tissue between a first jaw member and a second jaw member of a suturing apparatus, wherein a portion of a suture is engaged by a proximal portion of the first jaw member; pivoting a distal portion of the first jaw member relative to a distal portion of the second jaw member to wrap the suture around the tissue; passing, with a needle of the suturing apparatus, a length of the suture through the tissue; and engaging, with a retention member at the distal portion of the first jaw member, the length of suture to remove the length of the suture from the needle.

The method of the fifth aspect may have one or more of the following features. The portion of the suture may be a loop, and the length of the suture may be a tail, the method may include passing the tail through the loop. The method may include receiving the length of the suture in a seat on a proximal portion of the second jaw member before passing the length through the tissue. The method may include cinching the suture around the tissue by pulling the suturing apparatus away from the tissue.

### BRIEF DESCRIPTION OF THE DRAWINGS

The present application is further understood when read in conjunction with the appended drawings. For the purpose of illustrating the subject matter, there are shown in the exemplary drawings of the subject matter; however, the presently disclosed subject matter is not limited to the specific methods, devices, and systems disclosed. In the drawings:

FIG. 1 illustrates an exemplary suture passer according to the present disclosure.

FIG. 2 illustrates a jaw assembly of the suture passer of FIG. 1.

FIG. 3 illustrates an exemplary suture to be used with the suture passer of FIGS. 1 and 2.

FIG. 4 illustrates the suture of FIG. 3 being loaded on the jaw assembly of the suture passer of FIGS. 1 and 2.

FIG. 5 illustrates the suture passer of FIGS. 1, 2, and 4 loaded with the suture with the jaw assembly in a closed configuration.

FIG. 6 illustrates the suture passer of FIGS. 1, 2, 4, and 5 loaded with the suture with the jaw assembly in an open configuration.

FIG. 7 illustrates another view of the suture passer of FIGS. 1, 2, and 4-6 loaded with the suture and in the closed configuration.

FIG. 8 illustrates the suture passer of FIGS. 1, 2, and 4-7 loaded with the suture and receiving tissue received in the jaw assembly.

FIG. 9 illustrates the suture passer of FIGS. 1, 2, and 4-8 loaded with the suture with a needle in an extended configuration through the tissue.

FIG. 10 illustrates the suture passer of FIGS. 1, 2, and 4-9 with the needle in an extended configuration and the tissue removed for clarity.

FIG. 11 illustrates the suture passer of FIGS. 1, 2, and 4-10 with the suture engaged by a retention member.

FIG. 12 illustrates the suture passer of FIGS. 1, 2, and 4-11 with the suture engaged by the retention member and the jaw assembly in the open configuration.

FIG. 13 illustrates the suture passer of FIGS. 1, 2, and 4-12 being pulled from the tissue to cinch the suture around the tissue.

FIG. 14 illustrates a method of suturing tissue with the suture passer of FIGS. 1, 2, and 4-13.

Aspects of the disclosure will now be described in detail with reference to the drawings, wherein like reference numbers refer to like elements throughout, unless specified otherwise.

### DETAILED DESCRIPTION

The present disclosure provides a suture passer and techniques for surgical repairs such as arthroscopic surgeries including a tenotomy and a tenodesis. The suture passer may be configured to perform a Loop ‘N’ Tack™ stitch (as produced by Arthrex) in a single process. The suture passer may have a jaw assembly configured to grasp a soft tissue while looping a first portion of a suture at least partially around the soft tissue. The suture may be wrapped around the distal portion of the jaw assembly and held in tension with a cleat on the suture passer to facilitate insertion and looping the suture around the soft tissue. The distal portion of the jaw assembly may include a distal channel to a side of the suture retainer to prevent interference of the suture wrapped around the distal portion with the suture retainer. The jaw assembly may be inverted, such that a first jaw may pivot a distal connection with the second jaw to allow the suture passer to capture the soft tissue in tight spaces.

The suture passer may also have a needle configured to pass a second portion of the suture through the soft tissue. The first portion of the suture may include a fixed loop and the second portion may include a tail. The needle may push the tail of the suture through the fixed loop to secure the suture in the Loop ‘N’ Tack™ stitch. A suture retainer in the distal portion of the suture passer may retain the tail in the advanced positioned during removal of the suture passer from the soft tissue. Pushing the suture with the needle and capturing the suture with the suture retainer may prevent the needle from capturing the soft tissue or dropping the suture. The suture may then be tightened or cinched around the tissue as the suture passer pulled from the soft tissue, and the tail may then be secured to a bone tissue with a bone anchor.

Thus, the suture passer may engage the soft tissue with the jaw assembly while looping the suture at least partially around the soft tissue. The suture passer may further pass the needle that carries the tail of the suture through the fixed loop of the suture and through the soft tissue. The suture passer may then secure the tail of the suture to the suture retainer at the distal portion of the suture passer. Therefore, the suture passer may form the Loop ‘N’ Tack™ stitch without the need of multiple instruments, improving efficiency in the procedure. The suture passer may further eliminate the need for additional preparation of the soft tissue.

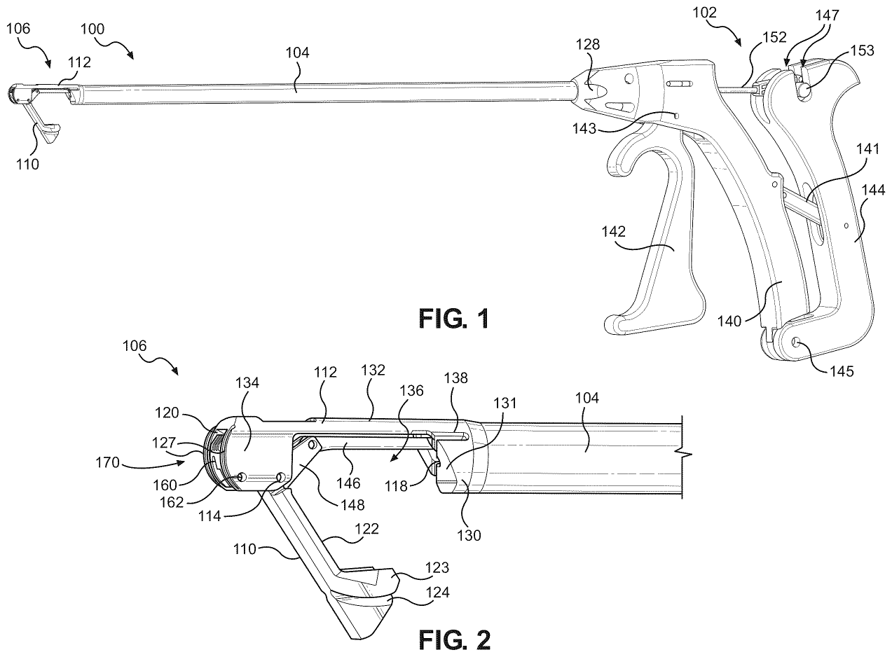

FIG. 1 illustrates a suture passer 100. The suture passer 100 may include a handle assembly 102, a shaft member 104 extending distally from the handle assembly 102, and a jaw assembly 106 at the distal end of the shaft member 104. The handle assembly 102 may actuate the jaw assembly 106 between an open configuration and a closed configuration.

The jaw assembly 106 may include a first jaw member 110 and a second jaw member 112, such that the first jaw member 110 may be configured to move relative to the second jaw member 112 between the open configuration and the closed configuration. The first jaw member 110 may pivot relative to the second jaw member 112, and the second jaw member 112 may be fixed relative to shaft member 104. A distal portion 121 of the first jaw member 110 may be pivotably attached to a distal portion 134 of the second jaw member 112 at a pivot point 114 in an inverted configuration.

The first jaw member 110 may be configured to pivot relative to the second jaw member 112 at the pivot point 114 between the open configuration and the closed configuration. The first jaw member 110 may be configured to pivot in a lateral plane relative to the second jaw member 112. The lateral plane may extend transverse to a longitudinal axis of the shaft member 104. The pivot point 114 may be formed by a pin connecting the distal portion 121 of the first jaw member 110 and the distal portion 134 of the second jaw member 112. The first jaw member 110 may have a pivoting arm 122 extending proximally from the distal portion 121 and a protrusion 123 forming a proximal portion of the arm 122. The protrusion 123 may be configured to secure and position the suture 20.

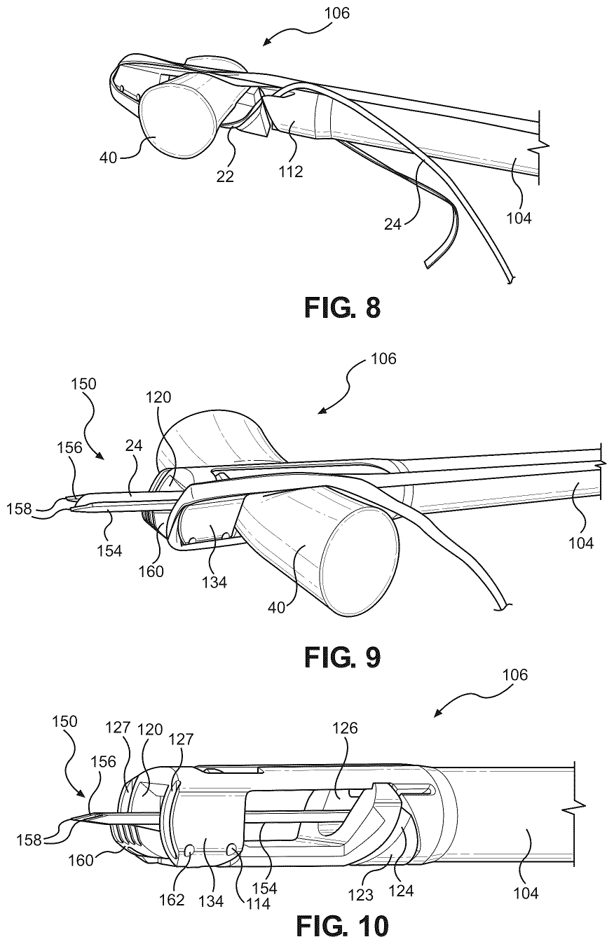

The second jaw member 112 may be an integral extension of the shaft member 104. The second jaw member 112 may be substantially C-shaped with a proximal portion 130, an intermediate portion 132, and the distal portion 134. The intermediate portion 132 may have a width less than a width of each of the proximal portion 130 and the distal portion 134 to define a recess 136. The recess 136 may receive tissue and the first jaw member 110 when the jaw assembly 106 is in the closed configuration. The jaw assembly 106 may be configured to wrap the suture 20 at least partially around a soft tissue 40 in the closed configuration, as illustrated in FIG. 8.

The handle assembly 102 may have a fixed handle member 140, a first trigger member 142, and a second trigger member 144. The fixed handle member 140 may be fixed to a proximal portion of the shaft member 104.

The first trigger member 142 may be movable relative to the fixed handle member 140 to actuate the jaw assembly 106 between the open configuration and the closed configuration. The first trigger member 142 may be configured to pivot relative to the fixed handle member 140 at a pivot point 143 to longitudinally translate an actuation shaft 146 through a first lumen of the shaft member 104. The pivot point 143 may be formed by a pin connecting the fixed handle member 140 and the first trigger member 142. A link 148 may be connected between the actuation shaft 146 and the first jaw member 110. A proximal portion of the link 148 may be pivotably connected to the distal portion of the actuation shaft 146. A distal portion of the link 148 may be pivotably connected to the distal portion 121 of the first jaw member 110. Longitudinal translation of the actuation shaft 146 may rotate and/or translate the link 148 to cause the first jaw member 110 to pivot relative to the second jaw member 112 between the open configuration and the closed configuration.

The second trigger member 144 may be movable relative to the fixed handle member 140 to longitudinally translate a needle 150 through a second lumen of the shaft member 104, as illustrated in FIGS. 9 and 10. A proximal opening 118 of the jaw assembly 106 may be formed by the distal end of the second lumen and through the proximal portion 130 of the second jaw member 112. The needle 150 may be attached to a needle shaft 152 connected to the second trigger member 144. For example, the needle shaft 152 may include one or more pins 153 configured to be received in one or more slots 147 of the second trigger member 144. Each pin 153 of the needle shaft 152 may slide in the slot 147 of the second trigger member 144 as the second trigger member 144 pivots relative to the fixed handle member 140 and the needle shaft 152 translates along a longitudinal axis of the shaft member 104. The needle shaft 152 may be an integral extension of the needle 150. The second trigger member 144 may be configured to pivot relative to the fixed handle member 140 at a pivot point 145 to longitudinally advance or retract the needle shaft 152 to advance or retract the needle 150 between a retracted configuration and an extended configuration. The pivot point 145 may be formed by a pin connecting the fixed handle member 140 and the second trigger member 144. A position retention arm 141 may be configured to control movement of the second trigger member 144 relative to the fixed handle member 140. The position retention arm 141 may include a retention device for preventing the needle shaft 152 from moving from a partially actuated position. Further discussion of the handle assembly 102 is provided in U.S. Pat. No. 9,078,650, the entire disclosure of which is incorporated herein by reference.

In the retracted configuration, a distal portion 154 of the needle 150 may be retracted into the second lumen of the shaft member 104 to prevent inadvertent puncturing, as illustrated in FIGS. 1 and 2. In the extended configuration, the distal portion 154 of the needle 150 may extend through the proximal opening 118, out of the second lumen of the shaft member 104, and through the jaw assembly 106, as illustrated in FIGS. 9 and 10. As further illustrated in the extended position, the distal portion 154 may extend through a distal opening 120 in the distal portion 134 of the second jaw member 112 and past a retention member 160. The needle 150 may be a straight needle configured to extend from the handle assembly 102, through the shaft member 104, and through the jaw assembly 106. As further illustrated in FIGS. 9 and 10, the distal portion 154 may have a groove 156 between first and second prongs 158. The groove 156 may be configured to receive the suture 20 as the needle 150 is advanced through and across the jaw assembly 106, and the first and second prongs 158 may be configured to puncture the soft tissue 40 to pass the suture 20 therethrough, as illustrated in FIG. 9. The configuration of the needle 150 pushing the suture 20 to be captured by the retention member 160 may create a secure exchange and retention of the suture 20, limiting the possibility that the suture 20 is dropped during the suturing process.

The first jaw member 110 may be a lower jaw member and the second jaw member 112 may be an upper jaw member when the user handles the suture passer 100 to facilitate grasping of the soft tissue 40. For example, the first jaw member 110 may be on the same side of the longitudinal axis of the shaft member 104 as the fixed handle member 140, the first trigger member 142, and/or the second trigger member 144. The configuration may facilitate capturing the soft tissue 40 in limited intracorporeal space, such as during a shoulder tenotomy or tenodesis to repair a bicep tendon.

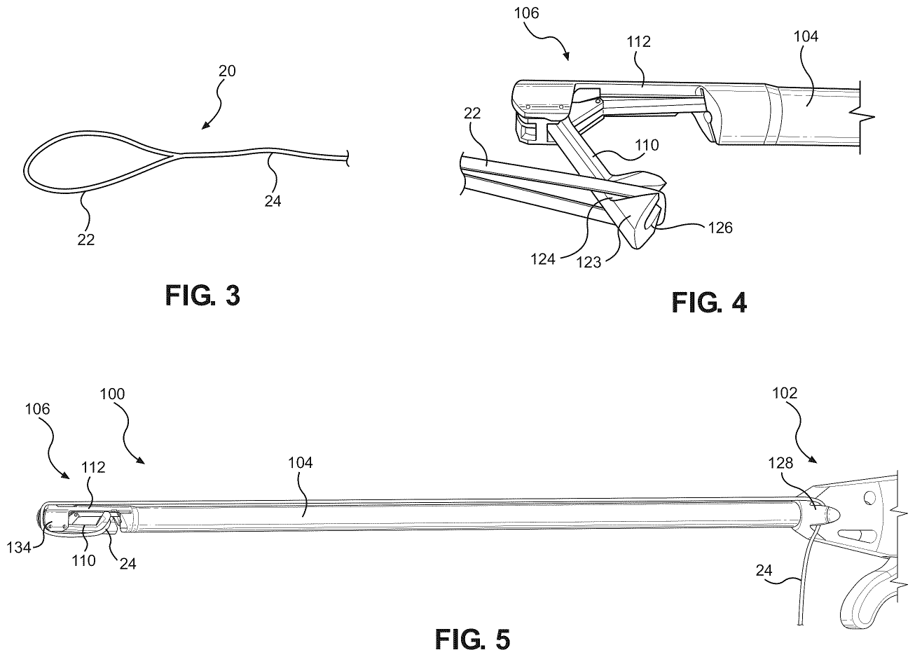

FIG. 3 illustrates an exemplary suture 20 to be loaded onto the suture passer 100 and applied to the soft tissue 40. The suture 20 may have a first portion 22 and a second portion 24. As illustrated, the first portion 22 may be or include a loop (e.g., a closed and/or fixed loop) and the second portion 24 may be or include a tail. The suture 20 may be formed of a strand of material that is looped to form the loop 22. The loop 22 may be a continuous, uninterrupted, flexible loop. The perimeter of loop 22 may be fixed. The loop 22 may be formed by splicing an end of the suture 20 into another portion of the suture 20. Additionally or alternatively, the loop 22 may be formed by fusing, gluing, bonding, joining, braiding, and/or interlinking the end portion of the suture 20. Thus, the suture 20 may be a single tail 24 terminating at a fixed, closed loop 22 at its distal end.

The suture 20 may be a single filament or fiber, or can include multiple continuous filaments, segments or regions of filaments that have different configurations (for example, different diameters and/or different compositions). The filament regions/segments may each be homogenous (i.e., formed of the same material) or may be a combination of homogenous and heterogenous (i.e., formed of a plurality of materials). Exemplary materials may include suture, silk, cotton, nylon, polypropylene, polyethylene, ultrahigh molecular weight polyethylene (UHMWPE), polyethylene terephthalate (PET), and polyesters and copolymers thereof, or combinations thereof. The suture 20 may have a round and/or flat cross-section. The suture 20 may be a FiberWire® suture, produced by Arthrex and disclosed in U.S. Pat. No. 6,716,234, the entire disclosure of which is incorporated herein by reference. Alternatively, the suture 20 may be a flat suture tape, such as FiberTape® produced by Arthrex.

As illustrated in FIG. 4, the suture 20 may be loaded onto the suture passer 100 by first securing the loop 22 to the first jaw member 110. The loop 22 of the suture 20 may be received in a lateral channel 124 in the protrusion 123 of the first jaw member 110. The lateral channel 124 may extend at least partially across an inner surface of the protrusion 123 to pull the loop 22 toward the intermediate portion 132 of the second jaw member 112 and into the recess 136 when in the closed configuration of the jaw assembly 106. The protrusion 123 may have a width in the lateral plane (that the first jaw member 110 pivots in) greater than a width of the arm 122 for the lateral channel 124 to align an opening of the loop 22 with the proximal opening 118 (and path of the needle 150). The lateral channel 124 may have a first portion on a first lateral side of the inner surface of the protrusion 123 and a second portion on a second lateral side of the inner surface of the of the protrusion 123, where the first portion and the second portion may be separated by a longitudinal channel 126 extending through the protrusion 123, as illustrated in FIGS. 4 and 10. The longitudinal channel 126 may be configured to receive the distal portion 154 of the needle 150 in the extended configuration, as further illustrated in FIG. 10. The lateral channel 124 may extend through the lateral sides of the protrusion 123 to prevent stress concentrations in the suture 20. As further illustrated in FIGS. 5 and 10, the sides of the lateral channel 124 may extend at an acute angle and/or be curved relative to the longitudinal axis of the suture passer 100 (with the jaw assembly 106 closed) to guide the suture 20 distally around the distal portion 134 of the jaw assembly 106 formed by the second jaw member 112.

As illustrated in FIGS. 5-7, the suture 20 may extend distally from the lateral channel 124 along an outer surface of the arm 122 of the first jaw member 110 and around the distal portion 134 of the second jaw member 112 and jaw assembly 106. As illustrated in FIG. 7, the distal portion 134 of the second jaw member 112 may have a distal surface 170 having the distal opening 120 extending therethrough. The distal surface 170 may be configured to receive the suture 20 when wrapped and guide the suture 20 laterally of the distal opening 120 to prevent interference with the retention member 160. For example, the distal surface 170 may have at least one distal channel 127 on a side of the distal opening 120 to receive and guide the suture 20. The at least one distal channel 127 may also prevent the suture 20 from sliding off of the distal portion 134 of the jaw assembly 106, ensuring alignment of the opening of the loop 22 with the proximal opening 118. As illustrated, the at least one distal channel 127 may include a first distal channel 127 on a first lateral side of the distal opening 120 and a second distal channel 127 on a second lateral side of the distal opening 120. The first and second distal channels 127 may extend substantially parallel to each other. The at least one distal channel 127 may extend along the lateral plane of the suture passer 100 that the first jaw member 110 pivots in. The user may choose one of the first and second distal channel 127 to thread the suture 20 around the distal portion 134 without interfering with the retention member 160. The at least one distal channel 127 may provide a path for the suture 20 distal of the recess 136 from a side of the first jaw member 110 toward a side of the intermediate portion 132 of the second jaw member 112.

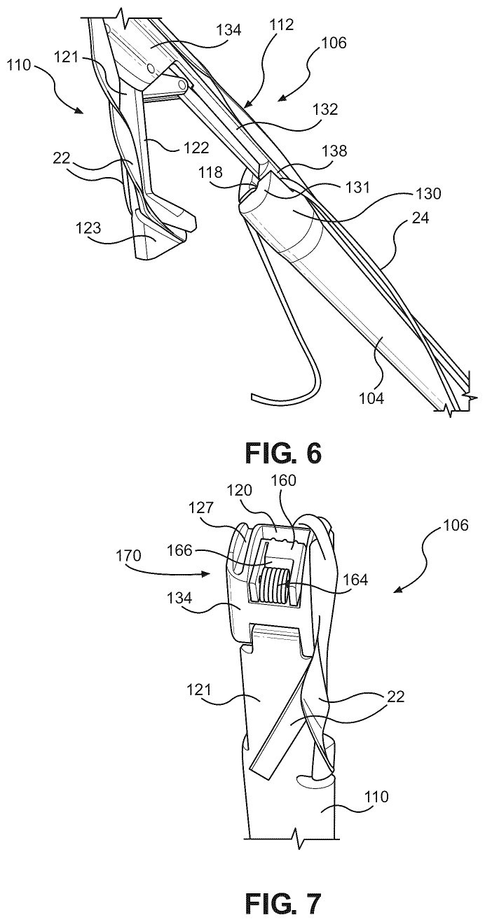

As further illustrated in FIGS. 5 and 6, the suture 20 may extend proximally from the distal portion 134 along an outer surface of the second jaw member 112, along the intermediate portion 132 and the proximal portion 130. The suture 20 may extend proximally along the shaft member 104 toward the handle assembly 102. The suture 20 may be tensioned along the path to keep the suture 20 in place during insertion in the body and/or actuation of the jaw assembly 106. The suture passer 100 may have at least one cleat 128 to engage the suture 20 and retain the tension to keep the suture 20. The at least one cleat 128 may be positioned on the shaft member 104 and/or the handle assembly 102. For example, as illustrated in FIG. 5, the at least one cleat 128 may be integrated into a distal portion of the handle assembly 102 proximal of the shaft member 104. The suture passer 100 may have a pair of cleats 128. The suture passer may have a first cleat 128 on a first side of the shaft member 104 and/or the handle assembly 102 and a second cleat (not shown) on a second side of the shaft member 104 and/or the handle assembly 102. The first and second cleat may be positioned on substantially the same longitudinal position of the shaft member 104 and/or the handle assembly 102. In some implementations, each of the cleats 128 may independently retain the suture 20 depending on the desired path of the suture 20. In other implementations, the suture 20 may be retained on both cleats 128.

Thus, the suture 20 may be held in place with the first portion 22 of the suture 20 (e.g., the loop) being received in the lateral channel 124 and the second portion 24 of the suture 20 (e.g., the tail) being received on the first cleat 128. The tension on the suture 20 provided by the at least one cleat 128 may provide suture management during insertion into the body and capturing the tissue. The suture management may prevent the suture 20 from unraveling or unwrapping from the distal end of the 134 of the jaw assembly 106, ensuring alignment of the opening of the loop 22 with the proximal opening 118. The suture management may also limit the possibility that the suture 20 gets snagged or caught with tissue during manipulation of the suture passer 100 into the body.

As illustrated in FIGS. 5 and 6, the second portion 24 (e.g., the tail) may be wrapped around or otherwise secured to the first cleat 128 and threaded or extended distally along the shaft member 104 to a distal-facing surface 131 of the proximal portion 130 proximal of the recess 136. Optionally, the suture 20 may then be threaded or extended proximally from the distal-facing surface 131 along the shaft member 104 and wrapped around or otherwise secured to the second cleat (not shown). The length of the suture 20 extending distally from the first cleat 128 to the distal-facing surface 131 and/or the length of the suture 20 extending proximally from the distal-facing surface 131 to the second cleat may have slack to allow the removal of the suture 20 from the at least one cleat 128 before actuation of the needle 150. Thus, the length of the suture 20 extending from the first jaw member 110 to the cleat 128 may have more tension (retained by the cleat 128) than the length of the suture 20 extending from the first cleat 128 to the distal-facing surface 131 and/or the length of the suture 20 extending from the distal-facing surface 131 to the second cleat.

The distal-facing surface 131 may have a seat 138 configured to releasably support a length of the second portion 24 of the suture 20. The seat 138 may include one or more slots in or through the distal-facing surface 131 extending transverse to a longitudinal axis of the shaft member 104. The seat 138 may be disposed at the proximal opening 118 of the second lumen to position the proximal portion across the path of the distal portion 154 of the needle 150. For example, the seat 138 may include a pair of slots on opposite sides of the proximal opening 118 and extending transverse to the longitudinal axis of the shaft member 104. The tail 24 of the suture may be releasably secured in the opposing slots of the seat 138 and have a length through or across the proximal opening 118. The slots of the seat 138 may be wedge shaped to secure the length of the tail 24 through a frictional and/or interference fit. The seat 138 may be recessed in the proximal portion 130 to allow the jaw assembly 106 to close without the first jaw member 110 interfering with the tail 24 received in the seat 138. The seat 138 may align and position the length of the tail 24 across the path of the needle 150, such that the needle 150 may receive the suture 20 in the groove 156 as the distal portion 154 of the needle 150 exits the second lumen.

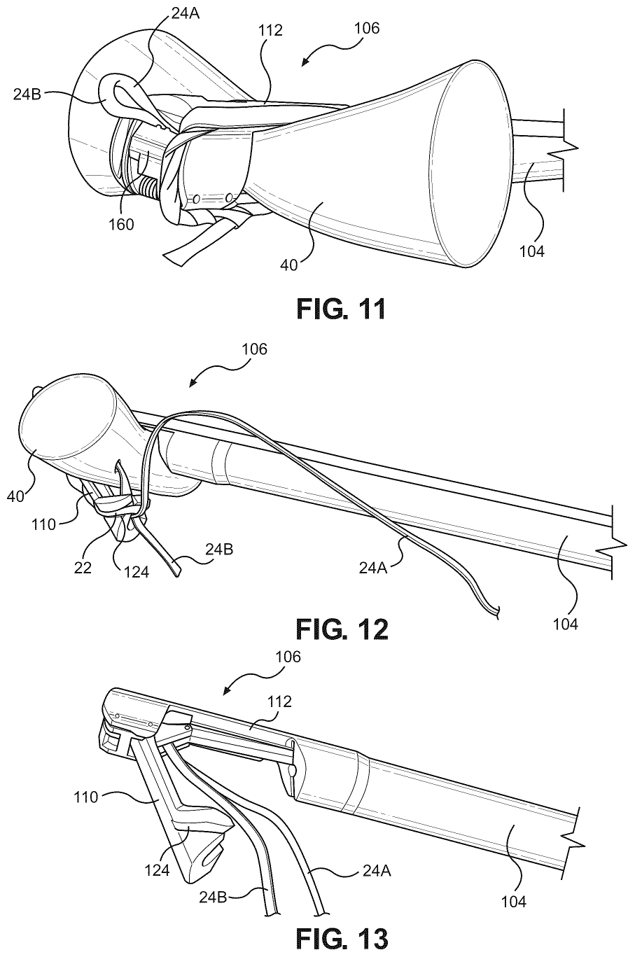

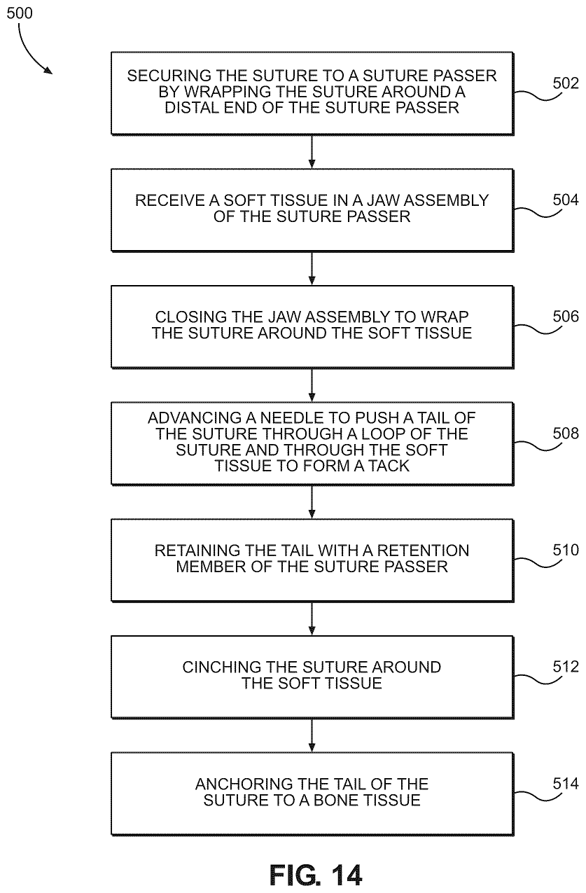

The needle 150 may push the tail 24 distally through the jaw assembly 106. The needle 150 may push the tail 24 through the loop 22 retained by the first jaw member 110. The needle 150 may further push the tail 24 through the tissue as the prongs 158 puncture the tissue. The needle 150 may further deflect the retention member 160 toward an open configuration (away from the longitudinal axis of the shaft member 104) and push the tail 24 through the distal opening 120, as illustrated in FIGS. 9 and 10. The needle 150 may then be retracted from the distal opening 120, and the retention member 160 may deflect back to a closed configuration (toward the longitudinal axis of the shaft member 104) to retain the tail 24 in the distal opening 120, as illustrated in FIG. 11.

The retention member 160 may be in the distal opening 120 and attached the second jaw member 112 at a pivot point 162. The retention member 160 may be a trap or a trapdoor, having a pivoting end securing to the distal portion 134 of the jaw assembly 106 and a free end configured to engage the suture 20. The pivoting end may be pivotably connected to the distal portion 134 of the second jaw member 112 at the pivot point 162. The retention member 160 may extend across the distal opening 120 and at least partially close the distal opening 120 when in the closed configuration, as illustrated in FIG. 2. The retention member 160 may at least partially open the distal opening 120 by engagement of the needle 150, as illustrated in FIGS. 9 and 10. The pivot point 162 may include a pivot pin extending across the second opening, where the retention member 160 may pivot between the closed configuration and the open configuration about the pivot pin. A spring 164 may engage the retention member 160 and the distal portion 134 of the second jaw member 112. The spring 164 may be configured to bias the retention member 160 toward the closed configuration. The spring 164 may be a separate component from the second jaw member 112 and/or the retention member 160, such that the spring 164 may be made of a metal and the second jaw member 112 and/or the retention member 160 may be made of a plastic. The spring 164 may be a helical spring or a leaf spring having a first end fixed to the retention member 160 and a second end fixed to the distal portion 134 of the second jaw member 112. The spring 164 may be coiled around the pivot pin and be received in a recess 166 of the retention member 160, as illustrated in FIG. 7. As further illustrated, the recess 166 may be substantially L-shaped to receive coils of the spring 164 and a leg secured to the retention member 160. The free end of the retention member 160 may have engaging members such as protrusions, teeth, and/or grooves configured to frictionally engage the suture 20 against a surface of the distal portion 134 to retain the suture in the distal opening 120. As further illustrated in FIG. 9, the needle 150 may push the suture 20 through the jaw assembly 106 and the distal opening 120, such that the tail 24 is folded over the distal portion 154 and forms a first length 24A and a second length 24B each extending through the loop 22 and secured by the retention member 160.

As illustrated in FIG. 12, the jaw assembly 106 may be opened with the suture 20 looped around and tacked through the soft tissue 40. The suture 20 may be secured to the suture passer 100 by the second portion 24 through the distal opening 120 being engaged by the retention member 160 and the first portion 22 being receive in the lateral channel 124 of the first jaw member 110. As illustrated in FIG. 13, the first portion 22 of the suture 20 may then be removed from the lateral channel 124 of the first jaw member 110, and the suture 20 may be retained by the retention member 160. The suture passer 100 may be further used to cinch the suture 20 around the tissue 40 with the suture 20 engaged by the retention member 160. The user may pull the jaw assembly 106 (with the suture 20 engaged by the retention member 160) away from the tissue 40 pulling the tail 24 further through the loop 22 and tensioning the suture 20 around the soft tissue 40.

The suture passer 100 may be used in an endoscopic or arthroscopic method 500 of repairing tissue, such as attaching the soft tissue 40 to a bone tissue (not shown) in a knotless repair. For example, the soft tissue 40 may be a tendon, and the tendon may be repaired in an arthroscopic bicep tenotomy or tenodesis with the suture passer 100.

In step 502, the method may include securing the suture 20 to the suture passer 100. The suture 20 may be secured by looping the first portion 22 into the lateral channel 124 of the first jaw member 110, as illustrated in FIG. 4. The suture 20 may be wrapped distally around the distal portion 134 of the second jaw member 112 into at least one distal channel 127 on a lateral side of the distal opening 120. The suture 20 may be extended proximally along the shaft member 104. The suture 20 may be tensioned and secured to one of the first cleat 128 to retain the tension. The suture 20 may then be extended distally along the shaft member 104 and secured in the seat 138 of the proximal portion 130 of the jaw assembly 106. The seat 138 may position the suture 20 across the pathway of the needle 150. The suture 20 may then be extended proximally from the seat 138 along the shaft member 104 and secured to the second cleat (not shown). The configuration of the at least one distal channel 127 and the at least one cleat 128 may allow the user to customize the suturing of the soft tissue 40. For example, in the application of a tenotomy or a tenodesis, the user may want to form the adjustable loop around the same side of the tack as the healthy tissue of the tendon 40 to ensure that the adjustable loop does not slip off of the tendon 40 after a diseased tissue side of the tendon 40 is cut.

In step 504, the suture passer 100 may be inserted into the body and the jaw assembly 106 may be opened to receive the soft tissue into the jaw assembly 106. In step 506, the jaw assembly 106 may be closed to wrap the suture 20 around the soft tissue in an adjustable loop. The jaw assembly 106 may be actuated to the closed configuration by the user pulling or squeezing the first trigger member 142 relative to the fixed handle member 140. Closing the jaw assembly 106 may be performed by pivoting the distal portion 121 of the first jaw member 110 relative to the distal portion 134 of the second jaw member 112 in an inverted configuration. In the closed configuration, the first jaw member 110 may position the loop 22 at the proximal opening 118 of the proximal portion 130 of the second jaw member 112. After the jaw assembly 106 is closed, the suture 20 may be removed from the cleat 128 to create more slack in the suture 20.

In step 508, the needle 150 may be advanced through the shaft member 104 out of the proximal opening 118 to engage the tail 24 of the suture to push the tail 24 through the loop 22 of the suture 20. The needle 150 may receive the tail 24 of the suture 20 in a groove 156 of the distal portion 154 as the needle exits the proximal opening 118. The needle 150 may be further advanced to push the tail 24 through the soft tissue 40 by puncturing the soft tissue 40 with the prongs 158. The needle 150 may further pass the tail 24 through the distal opening 120 and pivot the retention member 160 into the open configuration. Advancing the tail 24 through the loop 22 of the suture may close the adjustable loop of the suture 20 around the soft tissue 40. Further passing the tail 24 through the soft tissue 40 may create the tack. The needle 150 may be advanced by the user pulling or squeezing the fixed handle member 140 relative to the second trigger member 144. In step 510, the needle 150 may be retracted such that the spring 164 pivots the retention member 160 into the closed configuration to secure the tail 24 of the suture 20 in the distal opening 120. The retention member 160 may remove the tail 24 from the needle 150. The needle 150 may be retracted by the user pushing or releasing the fixed handle member 140 relative to the second trigger member 144.

In step 512, the suture 20 may be cinched. With the suture 20 engaged in the retention member 160, the jaw assembly 106 may be pulled away from the soft tissue 40. The engagement of the suture 20 with the retention member 160 may pull a remaining length of the tail 24 through the loop 22. The resultant construct of the suture 20 may form an adjustable loop around the soft tissue 40 that is tensionable by the tail 24 being tacked through the soft tissue 40. Further pulling of the tail 24 from the soft tissue 40 (with the suture passer 100 or after removing the suture 20 from the suture passer 100) may cinch the adjustable loop around the soft tissue 40 by tensioning the tail 24.

In step 514, the soft tissue 40 may be secured to the bone tissue by anchoring the tail 24 on a pre-drilled hole in the bone tissue. The tail 24 may be anchored to the bone tissue with a SwiveLock® or PushLock® anchor (both produced by Arthrex). For example, the tail 24 may be anchored to the bone tissue as disclosed in U.S. Pat. No. 10,881,388, the entire disclosure of which is incorporated herein by reference. Excess length of the tail 24 of the suture 20 may be cut before or after the tail 24 is anchored to the bone tissue. In the application of a tenotomy or a tenodesis, a diseased portion of the tendon 40 may be cut with the healthy portion of the tendon 40 being secured to the bone tissue. The customization of the suturing with the at least one distal channel 127 and/or the at least one cleat 128 may allow the cutting to be performed on an opposite side of the tack with respect to the adjustable loop, such that the tack prevents the adjustable loop from slipping off of the cut end of the tendon 40.

It will also be appreciated by those skilled in the art that modifications can be made without departing from the invention. Structural features of systems and apparatuses described herein can be replaced with functionally equivalent parts or omitted entirely. Moreover, it will be appreciated that features can be combined with each other without departing from the disclosure.

What is claimed is:

1. A suturing apparatus comprising:
a shaft member having a lumen;
a first jaw member and a second jaw member configured to move between an open configuration and a closed configuration, wherein a distal portion of the first jaw member is pivotably connected to a distal portion of the second jaw member, and the distal portion of the second jaw member having a distal opening;
a needle configured to translate through the lumen of the shaft member and configured to carry a suture; and
a retention member in the distal opening of the second jaw member, wherein the retention member is configured to engage the suture and remove the suture from the needle.

2. The suturing apparatus of claim 1, further comprising:
a handle assembly; and
a cleat on a first side of the shaft member and/or the handle assembly, wherein the suture is configured to be wrapped around the distal portion of the second jaw member under tension and to engage the cleat to maintain the tension.

3. The suturing apparatus of claim 2, further comprising a second cleat on a second side of the shaft member and/or the handle assembly opposite of the first side, wherein the second cleat is configured to engage the suture.

4. The suturing apparatus of claim 1, further comprising a spring configured to bias the retention member in a closed configuration.

5. The suturing apparatus of claim 1, wherein the suture is configured to be wrapped around the distal portion of the second jaw member on a lateral side of the retention member.

6. The suturing apparatus of claim 5, wherein a proximal portion of the first jaw member has a lateral channel configured to receive the suture, and a longitudinal channel configured to receive the needle.

7. The suturing apparatus of claim 1, wherein the second jaw member has a proximal portion having a seat configured to receive the suture.

8. The suturing apparatus of claim 1, wherein the needle has a groove between a first prong and a second prong, and the groove is configured to receive the suture.

9. A suturing system comprising:
the suture apparatus of claim 1; and
the suture having a loop and a tail, wherein the loop of the suture is engaged by a proximal portion of the first jaw member, the suture is wrapped around the distal portion of the second jaw member, and the needle engages a length of the tail and feeds the length of the tail through the loop of the suture.

10. A suturing apparatus comprising:
a shaft member having a lumen;
a first jaw member and a second jaw member configured to pivot between an open configuration and a closed configuration, wherein a distal portion of the second jaw member has a distal opening;
a needle configured to translate through the lumen of the shaft member and configured to carry a suture; and
a retention member in the distal opening of the second jaw member, wherein the retention member is configured to engage the suture and remove the suture from the needle,
wherein a distal surface of the first jaw member has at least one distal channel configured to receive a length of the suture.

11. The suturing apparatus of claim 10, wherein the first jaw member is configured to pivot in a lateral plane relative to the second jaw member, and the at least one distal channel extends along the lateral plane.

12. The suturing apparatus of claim 10, wherein the at least one distal channel includes a first distal channel on a first lateral side of the distal opening and a second distal channel on a second lateral side of the distal opening.

13. The suturing apparatus of claim 10, wherein a proximal portion of the first jaw member has a lateral channel configured to receive the suture, and a longitudinal channel configured to receive the needle.

14. The suturing apparatus of claim 10, wherein the second jaw member has a proximal portion having a seat configured to receive the suture.

15. The suturing apparatus of claim 10, further comprising a spring configured to bias the retention member in a closed configuration.

16. A suturing system comprising:
the suturing apparatus of claim 10; and
the suture having a loop and a tail, wherein the loop of the suture is engaged by a proximal portion of the first jaw member, the suture is wrapped around a distal portion of the second jaw member, and the needle engages a length of the tail and feeds the length of the tail through the loop of the suture.

17. A suturing system comprising:
a suture;
a suturing apparatus comprising:
a shaft member having a lumen;
a first jaw member and a second jaw member configured to pivot between an open configuration and a closed configuration, wherein a distal portion of the first jaw member is connected to a distal portion of the second jaw member; and
a needle configured to translate through the lumen of the shaft member and configured to carry the suture,
wherein the suture is engaged by the first jaw member, the suture is wrapped around the distal portion of the second jaw member.

18. The suturing system of claim 17, wherein the suture has a loop and a tail, wherein the loop of the suture is engaged by a proximal portion of the first jaw member, and the needle engages a length of the tail and feeds the length of the tail through the loop.

19. A suturing system comprising:
a suture having a loop and a tail;
a suturing apparatus comprising:
a shaft member having a lumen;
a needle configured to translate through the lumen of the shaft member;
a first jaw member and a second jaw member configured to move between an open configuration and a closed configuration, wherein a distal portion of the first jaw member is pivotably connected to a distal portion of the second jaw member, and the distal portion of the second jaw member has a distal opening; and
a retention member in the distal opening of the second jaw member, wherein the retention member is configured to engage the suture and remove the suture from the needle,
wherein the loop of the suture is engaged by a proximal portion of the first jaw member, and a length of the tail is positioned at an opening of the lumen of the shaft member, and
wherein the first jaw member, when in the closed configuration, is configured to align an opening of the loop with the opening of the lumen, the needle is configured to engage the length of the tail and feed the length of the tail through the loop, and the retention member is configured to secure the length of the tail.

20. A method comprising:
receiving a tissue between a first jaw member and a second jaw member of a suturing apparatus, wherein a portion of a suture is engaged by a proximal portion of the first jaw member;
pivoting a distal portion of the first jaw member relative to a distal portion of the second jaw member to wrap the suture around the tissue;
passing, with a needle of the suturing apparatus, a length of the suture through the tissue; and
engaging, with a retention member at the distal portion of the first jaw member, the length of suture to remove the length of the suture from the needle.

21. The method of claim 20, wherein the portion of the suture is a loop, and the length of the suture is a tail, the method further comprising passing the tail through the loop.

22. The method of claim 20, further comprising receiving the length of the suture in a seat on a proximal portion of the second jaw member before passing the length through the tissue.

23. The method of claim 20, further comprising cinching the suture around the tissue by pulling the suturing apparatus away from the tissue.
